(19) United States
(12) Patent Application Publication
(10) Pub. No.: US 2022/0346336 A1
(43) Pub. Date: Nov. 3, 2022

(54) GRAFTED CANNABIS PLANTS AND RELATED METHODS FOR PRODUCING GRAFTED CANNABIS PLANTS

(71) Applicant: ANANDIA LABORATORIES INC., Vancouver (CA)

(72) Inventors: Jonathan Page, Vancouver (CA); Gregory Baute, Comox (CA); John Mackenzie, Comox (CA); Kasi Williams, Vancouver (CA); Erin Gilchrist, Vancouver (CA)

(73) Assignee: ANANDIA LABORATORIES INC., Vancouver, BC (CA)

(21) Appl. No.: 17/621,196

(22) PCT Filed: Jun. 19, 2020
(86) PCT No.: PCT/CA2020/050852

§ 371 Date: Dec. 20, 2021

Related U.S. Application Data
(60) Provisional application No. 62/864,261, filed on Jun. 20, 2019.

(51) Int. Cl. A01H 4/00; A01H 6/28; A01G 2/30

(52) U.S. Cl. CPC A01H 4/005; A01H 6/28; A01G 2/30

(57) ABSTRACT

Grafted cannabis plants and related methods for producing a grafted cannabis plant are provided. In some embodiments, a scion is provided from a first cannabis plant having at least one of a first desired phenotype and a first desired genotype and a rootstock is provided from a second cannabis plant having at least one of a second desired phenotype and a second desired genotype, the rootstock compatible with the scion. The scion may be grafted onto the rootstock to form the grafted plant. In some embodiments, the scion is from a drug-type cannabis plant and the rootstock is from a hemp plant. The grafted cannabis plant may have at least one enhanced plant trait, enhanced harvest trait, and/or emergent property. Related cannabis plant materials and cannabis-derived products are also provided.

### RELATED APPLICATION

The present application claims priority to U.S. Provisional Patent Application No. 62/864,261, filed Jun. 20, 2019, the entire contents of which are incorporated by reference herein.

### FIELD OF THE DISCLOSURE

The present disclosure relates to grafted plants. More particularly, the present disclosure relates to grafted cannabis plants and methods for producing grafted cannabis plants.

### BACKGROUND

For some plant types, commercially available seeds may lack the genetic uniformity for large scale propagation. However, clonally propagated plants may have different growth and development characteristics compared to genetically similar plants grown from seed.

One plant type that is conventionally propagated clonally is cannabis. Cannabis refers to plants of the Cannabis genus. The Cannabis genus is generally understood to comprise one species, Cannabis sativa L., although some botanical authorities also recognize Cannabis indica and Cannabis ruderalis. Cannabis plants produce a variety of chemical compounds, including a unique family of terpeno-phenolic compounds called cannabinoids. Cannabinoids may be extracted from cannabis plants and used for a variety of commercial purposes, for example, in pharmaceutical products to treat various medical conditions. Cannabinoids with pharmaceutical applications include Δ⁹-tetrahydrocannabinol (THC) and cannabidiol (CBD).

Some growers may use grafting as an alternative method to propagate plants. Grafting is a procedure to splice two separate plants together by joining wounded stem tissues and promoting the formation of vasculature connections between the two plants. The upper part of the combined plant is called the scion while the lower part is called the rootstock. Some cannabis growers have grafted multiple cannabis scions onto a single cannabis rootstock to allow growth of multiple cannabis genotypes on a single plant.

In a 1975 report by Crombie and Crombie, inter-racial grafts were produced between high and low tetrahydrocannabinol (THC) genotypes of Cannabis sativa as well as cross-grafts between Cannabis sativa andHumulus species (Crombie and Crombie, “Cannabinoid formation in Cannabis sativa grafted inter-racially, and with two Humulus species”, Phytochemistry 1975 Vol 14, pp 409-412). Briefly, seedlings of the two plants to be grafted were grown side-by-side and cut portions of each stem were then joined together. The authors found that high THC cannabis scions grafted to low THC cannabis rootstock (and vice versa) showed minimal change in cannabinoid content or composition. On the other hand, grafting of cannabis scions onto Humulus (i.e. hop) rootstock appeared to stimulate total cannabinoid production.

However, worldwide cannabis cultivation has increased dramatically since the 1975 Crombie and Crombie report and modern cannabis cultivars bear little resemblance to those from over forty years ago. It is unknown if beneficial effects can be achieved by grafting cannabis scions onto different rootstocks.

### SUMMARY

In one aspect, there is provided a method for producing grafted cannabis plant, the method comprising: providing a scion from a first cannabis plant, the first cannabis plant having at least one of a first desired phenotype and a first desired genotype; providing a rootstock from a second cannabis plant, the second cannabis plant having at least one of a second desired phenotype and a second desired genotype, the rootstock compatible with the scion; and grafting the scion onto the rootstock.

In some embodiments, the first desired phenotype comprises at least one of a first biochemical phenotype, a first morphological phenotype, a first photoperiod or autoflowering phenotype, and a first production phenotype.

In some embodiments, the first biochemical phenotype comprises a desired cannabinoid content.

In some embodiments, the desired cannabinoid content comprises at least one of high THC content, high CBD content, and high cannabinoid content.

In some embodiments, the first morphological phenotype comprises at least one of plant size, plant height, stem thickness, flower number, flower size, flower color, flower density, trichome density, branching number, branching degree, branching architecture, and internode length.

In some embodiments, the first production phenotype comprises at least one of growth rate, yield, stress tolerance, and plant vigor.

In some embodiments, the second desired phenotype comprises as least one of a second biochemical phenotype, a second morphological phenotype, a second photoperiod or autoflowering phenotype, and a second production phenotype.

In some embodiments, the second morphological phenotype comprises at least one of plant size, plant height, stem thickness, root structure morphology, root thickness, and rooting quality.

In some embodiments, the second production phenotype comprises at least one of growth rate, yield, stress tolerance, plant vigor, and nutrient uptake.

In some embodiments, the first desired phenotype comprises the first biochemical phenotype and the second desired phenotype comprises at least one of the second morphological phenotype, and the second production phenotype.

In some embodiments, the first desired genotype comprises a genotype associated with a drug-type variety or cultivar.

In some embodiments, the drug-type variety or cultivar is Cannabis sativa L. subspecies sativa or Cannabis sativa L. subspecies indica.

In some embodiments, the drug-type variety or cultivar is a hybrid of Cannabis sativa L. subspecies sativa and Cannabis sativa L. subspecies indica.

In some embodiments, the second desired genotype comprises a genotype associated with a hemp variety or cultivar.

In some embodiments, at least one of the first and second cannabis plants is clonally propagated.

In some embodiments, at least one of the first and second cannabis plants is seed-grown.

In some embodiments, the first cannabis plant is clonally propagated and the second cannabis plant is seed-grown.

In some embodiments, the first cannabis plant is seed-grown and the second cannabis plant is clonally propagated.

In another aspect, there is provided a method for producing a grafted cannabis plant, comprising: providing a scion from a drug-type cannabis plant; providing a rootstock from a hemp plant, the rootstock compatible with the scion; and grafting the scion onto the rootstock.

In some embodiments, the drug-type cannabis plant is a cultivar of Cannabis sativa L. subspecies sativa or Cannabis sativa L. subspecies indica.

In some embodiments, the drug-type cannabis plant is a hybrid of Cannabis sativa L. subspecies sativa and Cannabis sativa L. subspecies indica.

In some embodiments, at least one of the drug-type cannabis plant and the hemp plant is clonally propagated.

In some embodiments, at least one of the drug-type cannabis plant and the hemp plant is seed-grown.

In some embodiments, the drug-type cannabis plant is clonally propagated and the hemp plant is seed-grown.

In some embodiments, the drug-type cannabis plant is seed-grown and the hemp plant is clonally propagated.

In another aspect, there is provided a grafted cannabis plant produced by any embodiment of the methods described herein.

In some embodiments, the grafted cannabis plant exhibits at least one of: an enhanced plant trait, an enhanced harvest trait, and an emergent property.

In some embodiments, the emergent property is not present in the first and second cannabis plants.

In some embodiments, the emergent property is substantially different than the first desired phenotype and the second desired phenotype.

In another aspect, there is provided a cannabis plant material obtained from any embodiment of the grafted plants described herein.

In some embodiments, the cannabis plant material comprises dried flower.

In another aspect, there is provided a cannabis-derived product generated from any embodiment of the cannabis plant material described herein, wherein the cannabis-derived product comprises one or more of an extract, a concentrate, a resin, an isolate, and an oil.

In another aspect, there is provided a food product comprising any embodiment of the cannabis-derived product described herein.

In another aspect, there is provided a beverage product comprising any embodiment of the cannabis-derived product described herein.

In another aspect, there is provided a cosmetic product comprising any embodiment of the cannabis-derived product described herein.

In another aspect, there is provided a topical product comprising any embodiment of the cannabis-derived product described herein.

In another aspect, there is provided a pharmaceutical product comprising any embodiment of the cannabis-derived product described herein.

In another aspect, there is provided a veterinary product comprising any embodiment of the cannabis-derived product described herein.

In another aspect there is provided a vaporizable product comprising any embodiment of the cannabis-derived product described herein.

Other aspects and features of the present disclosure will become apparent, to those ordinarily skilled in the art, upon review of the following description of the specific embodiments of the disclosure.

### BRIEF DESCRIPTION OF THE DRAWINGS

Some aspects of the disclosure will now be described in greater detail with reference to the accompanying drawings. In the drawings:

FIG. 1 is a flowchart of an example method for producing a grafted cannabis plant, according to some embodiments;

FIG. 2 is a flowchart of another example method, according to some embodiments;

FIG. 3 is a photograph showing grafted Blue Dream cannabis plants, according to some embodiments, at 15 days after grafting (right); clonally propagated cannabis plants grown for the same time period are shown for comparison (left);

FIG. 4 is a photograph showing twelve of the grafted Blue Dream cannabis plants of FIG. 3 at 12 days after the induction of the flowering photoperiod (right); twelve clonally propagated cannabis plants grown for the same time period are shown for comparison (left);

FIG. 5 is a photograph showing the twelve grafted Blue Dream cannabis plants of FIG. 4 at 64 days after the induction of the flowering photoperiod (right); the twelve clonally propagated cannabis plants grown for the same time period are shown for comparison (left);

FIG. 6 is a photograph showing two of the grafted Blue Dream cannabis plants of FIG. 5 at 64 days after the induction of the flowering photoperiod (left); two clonally propagated cannabis plants grown for the same time period are shown for comparison (right);

FIG. 7 is a line graph showing the average plant height (cm) of clonally propagated and grafted Blue Dream cannabis plants between 3 and 73 days post-transplantation and 17 to 87 days post-grafting, respectively; error bars denote standard error of the mean;

FIG. 8 is a line graph showing cumulative duration of flood-and-drain irrigation (minutes) required to maintain similar soil moisture levels for clonally propagated and grafted Blue Dream cannabis plants between 3 and 73 days post-transplantation and 17 to 87 days post-grafting, respectively;

FIG. 9 is a bar graph showing the average weight (g) of dry floral material, fresh floral material, and vegetative material of clonally propagated and grafted Blue Dream cannabis plants at harvest; error bars denote standard error of the mean;

FIG. 10 is a bar graph showing the average weight percent (w/w) THC to total cannabinoids of dry flower material from clonally propagated and grafted Blue Dream cannabis plants at harvest; error bars denote standard error of the mean;

FIG. 11 is a bar graph showing the average per-plant total cannabinoid and THC content (mg/plant) of clonally propagated and grafted Blue Dream cannabis plants at harvest; error bars denote standard error of the mean;

FIG. 12 is a bar graph showing the average terpene content (% dry weight) of clonally propagated and grafted cannabis Blue Dream plants at harvest; error bars denote standard error of the mean;

FIG. 13 is a bar graph showing average percent Δ⁹-tetrahydrocannabinolic acid (THCA) content (% dry weight) of clonally propagated and grafted Purple Kush cannabis plants;

FIG. 14 is a bar graph showing average weight (g) of dry floral material, fresh floral material, and vegetative material of clonally propagated and grafted Purple Kush cannabis plants at harvest; error bars denote standard error of the mean;

FIG. 15 is a bar graph showing average harvest index (proportion of total plant weight that is flower) of clonally propagated and grafted Purple Kush cannabis plants; error bars denote standard error of the mean;

FIG. 16 is a bar graph showing average weight percent (w/w) THCA to total cannabinoids of dry flower material from clonally propagated and grafted Purple Kush cannabis plants at harvest; error bars denote standard error of the mean; and

FIG. 17 is a bar graph showing average per plant THCA content (mg/plant) of dried flower from clonally propagated and grafted Purple Kush cannabis plants at harvest; error bars denote standard error of the mean.

### DETAILED DESCRIPTION OF THE EMBODIMENTS

Generally, the present disclosure provides a grafted cannabis plant and a related method for producing a grafted cannabis plant. In some embodiments, a scion is provided from a first cannabis plant having at least one of a first desired phenotype and a first desired genotype and a rootstock is provided from a second cannabis plant having at least one of a second desired phenotype and a second desired genotype, the rootstock compatible with the scion. The scion may be grafted onto the rootstock to form the grafted plant. In some embodiments, the scion is from a drug-type cannabis plant and the rootstock is from a hemp plant. In some embodiments, the grafted plant may exhibit improved plant traits, harvest traits, or both, compared to clonally propagated plants.

As used herein and in the appended claims, the singular forms of “a”, “an, and “the” include plural referents unless the context clearly dictates otherwise. For example, reference to “a plant” or “the plant” may also include a plurality of plants, including genetically similar or identical progeny of a single plant.

As used herein, a “grafted plant” or an “engrafted plant” may refer to a plant comprising at least one scion and at least one rootstock, wherein the scion is grafted onto the rootstock. As used herein, a “scion” may refer to the upper part of the grafted plant that is grafted onto the rootstock. The scion may comprise at least one stem part. The scion may further comprise at least one aerial part, including for example, leaves, flowers, and fruits. As used herein, “rootstock” may refer to the lower part of the plant onto which the scion is grafted. The rootstock may comprise at least one stem part and a root system. The root system may comprise a main tap root or a fibrous, branched root structure. As used herein, a “compatible” rootstock to a given scion may refer to a rootstock from a plant of sufficiently close genetic relationship to allow a vascular connection to form between the rootstock and the given scion. A compatible rootstock may be of the same family, the same genus, the same species, or the same variety or cultivar as the scion.

As used herein, “variety” and cultivar” may each be used to refer to a plant line that is substantially distinct, stable, and uniform in its characteristics when propagated. It will be understood to a person skilled in the art that individual plants of a given variety or cultivar may be genetically similar but may not be genetically identical.

As used herein, a “cannabis plant” may refer to a plant from the Cannabis genus. A grafted cannabis plant may comprise at least one of a scion and a rootstock from a plant of the Cannabis genus. The plant of the Cannabis genus may be Cannabis sativa L. In some embodiments, the cannabis plant may be Cannabis sativa subspecies sativa, Cannabis sativa subspecies indica, or Cannabis sativa subspecies ruderalis. Alternatively, some botanical authorities recognize Cannabis sativa, Cannabis indica, and Cannabis ruderalis as separate species. In some embodiments, the plant of the Cannabis genus may be a hybrid of two different subspecies/species. As used herein, “hybrid” may refer to a plant (or a variety or cultivar of plants) resulting from the crossing of parent plants from different varieties or cultivars, subspecies, or species.

In some embodiments, the grafted cannabis plant may comprise a scion from a first cannabis plant and a rootstock from a second cannabis plant.

In some embodiments, at least one of the first and second cannabis plants may be clonally propagated. As used herein, “clonally propagated” or “clone” may refer to a plant that has been produced from an individual parent plant by asexual reproduction. In some embodiments, the clonally propagated plant may be grown from a cutting from another cannabis plant. In other embodiments, the clonally propagated plant may be grown from one or more cloned cells in tissue culture.

In some embodiments, at least one of the first and second cannabis plants may be a seed-grown plant. As used herein, “seed-grown” may refer to a plant grown from a seed produced by sexual reproduction.

In some embodiments, the first cannabis plant may be clonally propagated and the second cannabis plant may be seed-grown. In other embodiments, the first cannabis plant may be seed-grown and the second cannabis plant may be clonally propagated. Alternatively, the first and second cannabis plants may each be clonally propagated or seed-grown.

In some embodiments, at least one of the first cannabis plant and the second cannabis plant may be selected based on a first desired phenotype and a second desired phenotype, respectively. As used herein, “phenotype” may refer to any observable plant trait or combination of plant traits. A “trait” in this context may refer to any feature, characteristic, quality, and/or attribute of the plant. A phenotype may be the result of a genetic component, an environmental component, or both. In some embodiments, the desired phenotype may comprise the presence of one or more specific plant traits. In other embodiments, the desired phenotype may comprise the absence of one or more specific plant traits. It will be understood to a person skilled in the art that certain phenotypes will be more relevant to the first cannabis plant (from which the scion is taken) than the second cannabis plant (from which the rootstock in taken) and vice versa.

In some embodiments, at least one of the first desired phenotype and the second desired phenotype may comprise a biochemical phenotype. As used herein, a “biochemical phenotype” may refer to any trait relating to the chemical composition of the plant. In some embodiments, the first biochemical phenotype may comprise a desired cannabinoid content in any part of the plant including, for example, the flowers and leaves. As used herein, “cannabinoid” may refer to any chemical compound capable of acting on a cannabinoid receptor in the human body. The desired cannabinoid content may comprise one or more of: the presence or absence of one or more cannabinoids; a total amount or concentration of one or more cannabinoids (e.g. an upper or lower threshold concentration and/or a concentration range); a ratio of one or more cannabinoids to one or more other cannabinoids; and any other relevant characteristic of the cannabinoid content of the cannabis plant.

In some embodiments, the desired cannabinoid content may comprise at least one of cannabidiol (CBD) and Δ⁹-tetrahydrocannabinol (THC). As used herein, “CBD” is intended to include cannabidiolic acid (CBDA) and any possible enantiomer or isomer of CBD or CBDA. As used herein, “THC” is intended to include Δ⁹-tetrahydrocannabinolic acid (THCA) and any possible enantiomer or isomer of THC or THCA. Within cannabis plants, CBD mainly occurs in the form CBDA and THC mainly occurs in the form of THCA. CBDA and THCA are converted to CBD and THC, respectively, when cannabis plant material is heated in a process known as decarboxylation.

In some embodiments, the desired cannabinoid content may comprise one or more of: cannabidiol (CBD), cannabidiolic acid (CBDA), Δ⁹-tetrahydrocannabinol (THC), Δ⁹-tetrahydrocannabinolic acid (THCA), cannabinol (CBN), cannabigerol (CBG), cannabigerolic acid (CBGA), (±)-cannabichromene (CBC), (±)-cannabichromenic acid (CBCA), cannabicyclol (CBL), cannabivarin (CBV), tetrahydrocannabivarin (THCV), tetrahydrocannabivarin acid (THCVA), cannabidivarin (CBDV), cannabidivarin acid (CBDVA), cannabigerovarin (CBGV), cannabigerovarin acid (CBGVA), cannabichromevarin (CBCV), any isomers or enantiomers thereof, and any other cannabinoid that can be present in cannabis plants.

In some embodiments, the desired cannabinoid content may comprise high cannabinoid content in any part of the plant. As used herein, “high-cannabinoid content” or a “high-cannabinoid plant” may refer to a plant having at least about 5% total cannabinoid content by dry weight in any part of the plant, including the leaves and flowers. In some embodiments, the desired cannabinoid content may comprise about 5% or greater, about 10% or greater, about 20% or greater, or about 30% or greater total cannabinoid content by dry weight.

In some embodiments, the desired cannabinoid content may comprise high THC content and/or high CBD content. As used herein, “high THC content” or a “high-THC plant” may refer to a plant having at least about 5% THC content by dry weight in any part of the plant, including the leaves and flowers. In some embodiments, the desired cannabinoid content may comprise about 5% or greater, about 10% or greater, or about 20% or greater THC by dry weight. As used herein, “high CBD content” or a “high-CBD plant” may refer to a plant having at least about 5% CBD by dry weight in any part of the plant, including the leaves and flowers. In some embodiments, the desired cannabinoid content may comprise about 5% or greater, about 10% or greater, or about 20% or greater CBD by dry weight.

In some embodiments, the desired cannabinoid content may comprise at least a legal limit THC by dry weight in any part of the plant, including the leaves and flowers. As used herein, a “legal limit”, when used in reference to THC content, may refer to a limit set by a regulatory authority in a given jurisdiction to distinguish between “drug-type” cannabis plants and hemp plants (described in more detail below). It will be understood by one skilled in the art that legal limits can differ between different jurisdictions and such limits can also be changed based on changes in regulations. In some embodiments, a legal limit comprises about 0.3% THC by dry weight in any part of the plant, including the leaves and flowers. In some embodiments, a legal limit comprises about 0.2% THC by dry weight in any part of the plant, including the leaves and flowers. In other embodiments, the desired cannabinoid content may comprise less than the legal limit THC by dry weight in any part of the plant, including the leaves and flowers.

In other embodiments, the desired cannabinoid content may comprise low cannabinoid content. As used herein, “low cannabinoid content”, or a “low-cannabinoid plant” may refer to a plant with less than about 5% total cannabinoid content by dry weight of any part of the plant, including the leaves and flowers. In some embodiments, the desired cannabinoid content may comprise low THC content and/or low CBD content. As used herein, “low THC content” or a “low-THC plant” may refer to a plant having less than about 5% THC by dry weight in any part of the plant, including the leaves and flowers. In some embodiments, the desired cannabinoid content may comprise about 5% or less, about 2% or less, about 1% or less THC by dry weight. As used herein, “low CBD content” or a “low-CBD plant” may refer to a plant having less than about 5% CBD by dry weight in any part of the plant, including the leaves and flowers. In some embodiments, the desired cannabinoid content may comprise about 5% or less, about 2% or less, or about 1% or less CBD by dry weight.

In other embodiments, the desired cannabinoid content may comprise high THC content and low CBD content (or vice versa) or any other desired content of one or more cannabinoids. In some embodiments, the desired cannabinoid content may comprise about a 1:1 ratio of THC content:CBD content.

In other embodiments, the biochemical phenotype may comprise a desired terpene content. The desired terpene content may comprise at least one of: the presence or absence of one or more terpenes; a total amount or concentration of one or more terpenes; a ratio of one or more terpenes to one or more other terpenes, and/or any other relevant characteristic of the terpene content of the cannabis plant.

As used herein, “terpene” may refer to isoprene-containing hydrocarbons and related oxygen-containing compounds such as alcohols, aldehydes or ketones (terpenoids) found in the essential oils of plant (e.g. cannabis plants). Non-limiting examples of terpenes include alpha-pinene, beta-pinene, camphene, sabinene, beta-myrcene, p-mentha-1,5-diene, (+)-3-carene, alpha-terpinene, limonene, eucalyptol, trans-beta-ocimene, beta-ocimene, gamma-terpinene, sabinene hydrate, fenchone isomers, terpinolene, linalool, fenchyl alcohol, camphor isomers, isopulegol, isoborneol, borneol isomers, hexahydrothymol, alpha-terpineol, gamma-terpineol, geranyl acetate, pulegone, geraniol, nerol, alpha-cedrene, trans-caryophyllene, alpha-humulene, valencene, cis-nerolidol, trans-nerolidol, caryophyllene oxide, guaiol, cedrol, and alpha-bisabolol.

In other embodiments, the biochemical phenotype may comprise a desired flavonoid content. The desired flavonoid content may comprise at least one of: the presence or absence of one or more flavonoids; a total amount or concentration of one or more flavonoids; a ratio of one or more flavonoids to one or more other flavonoids; and/or any other relevant characteristic of the flavonoid content of the cannabis plant.

As used herein, “flavonoid” may refer to a class of polyphenolic compounds found in plants. Non-limiting examples of flavonoids include cannflavin A, cannflavin B, cannflavin C, vitexin, isovitexin, apigenin, kaempferol, quercetin, luteolin, orientin and beta-sitosterol.

In other embodiments, at least one of the first desired phenotype and second desired phenotype may comprise a morphological phenotype. As used herein, a “morphological phenotype” may refer to any structural trait of the plant.

In some embodiments, the first desired phenotype comprises a first morphological phenotype. In some embodiments, the first morphological phenotype may comprise one or more of: plant size, plant height, stem thickness, flower number, flower size, flower color, flower density, trichome density, branching number, branching degree, branching architecture, internode length, and any other morphological phenotype relevant to the scion.

In some embodiments, the second desired phenotype may comprise a second morphological phenotype. In some embodiments, the second morphological phenotype may comprise one or more of: plant size, plant height, stem thickness, root structure morphology, root thickness, rooting quality, and any other morphological phenotype relevant to the rootstock. In some embodiments, the root structure morphology may comprise a single main tap root. In other embodiments, the root structure morphology may comprise a fibrous, branched root structure.

In other embodiments, at least one of the first desired phenotype and second desired phenotype may comprise a photoperiod phenotype. As used herein, “photoperiod” may refer to the day length to which a plant is exposed. As used herein, a “photoperiod phenotype” (also referred to as a “day length sensitive phenotype”) may refer to an ability of a plant to initiate flowering with the onset of reduced photoperiod (i.e. decreasing day length). Plants having a photoperiod phenotype may sense changes in day length and initiate flowering upon transition from longer days (longer day length) to shorter days (shorter day length). “Longer days” in this context may refer to day lengths of between about 16 hours and about 18 hours (i.e. between about 6 and 8 hours of darkness). “Shorter days” in this context may refer to day lengths of between about 12 hours and about 14 hours (i.e. between about 10 and 12 hours of darkness). However, the skilled person will understand that it is the change in the day length that is important to the initiation of flowering rather than the specific length of the “longer days” or “shorter days”.

In other embodiments, at least one of the first desired phenotype and second desired phenotype may comprise an autoflowering phenotype. As used herein, an “autoflowering phenotype” (also referred to as a “day length neutral phenotype”) may refer to an ability of a plant to initiate flowering with developmental age regardless of the photoperiod (or change thereto) to which the plant is exposed. For example, an autoflowering cannabis plant may flower at approximately 30 days post-germination without experiencing shorter day lengths.

In other embodiments, at least one of the first desired phenotype and second desired phenotype may comprise a production phenotype. As used herein, a “production phenotype” (also referred to as a “growth phenotype” or a “productivity phenotype”) may refer to any characteristic relating to growth and productivity of the plant.

In some embodiments, the first desired phenotype may comprise a first production phenotype. In some embodiments, the first production phenotype comprises at least one of growth rate, plant vigor, and yield. As used herein, “vigor” or “plant vigor” may refer to a capacity for growth and survival. As used herein, “yield” or “plant yield” may refer to the amount (e.g. weight or size) and/or quantity of tissues or organs produced per plant or per population of plants over a period of time. In some embodiments, the yield may comprise floral yield, i.e. the yield of flowers, flower buds, trichome heads, trichomes, seeds, etc.

In other embodiments, the second desired phenotype may comprise a second production phenotype. In some embodiments, the second production phenotype may comprise at least one of growth rate, plant vigor, yield, and nutrient uptake. As used herein, “nutrient uptake” may refer to a plant's ability to absorb nutrients from the soil (or other growth media) via the roots. For example, the nutrients may comprise one or more of nitrogen, potassium, phosphorus, and/or any other nutrient relevant to the plant.

In some embodiments, at least one of the first production phenotype and second production phenotype may comprise a stress tolerance phenotype. As used herein, “stress tolerance” or a “stress tolerance phenotype” may refer to an ability of a plant to reduce or avoid damage and/or loss of viability in the presence of an abiotic or biotic stress. In some embodiments, the stress tolerance phenotype may comprise at least one of disease tolerance, cold tolerance, and drought tolerance. As used herein, “disease tolerance” and “disease resistance” may be used interchangeably and may refer to an ability of a plant to prevent or suppress infection of a plant pathogen (for example (without limitation) mold and mildew) in all or part of the plant and/or an ability to reduce or avoid one or more disease symptoms. As used herein, “cold tolerance” may refer to an ability of a plant to reduce or avoid damage and/or remain viable at temperatures below the optimal temperature range for that plant. For example, a cannabis plant having a cold tolerant phenotype may survive at temperatures of about 20° C. or less, about 15° C. or less, or about 10° C. or less. As used herein, “drought tolerance” may refer to any ability of a plant to reduce or avoid damage and/or remain viable under moisture conditions lower than the optimal moisture level for that plant.

In other embodiments, the first and second desired phenotypes may comprise any other desirable phenotype or combination of phenotypes. It will be understood to one skilled in the art that many of the phenotypes described herein are multifactorial phenotypes that may be influenced by multiple individual traits of the plant. Moreover, it will be understood that some phenotypes may be related to or influenced by other phenotypes and therefore a plant having one desired phenotype may inherently have one or more other desired phenotypes. As one example, a plant having a high flower number will also likely have a high floral yield.

In some embodiments, the first desired phenotype may be different than the second desired phenotype. In some embodiments, the first desired phenotype may comprise a desired cannabinoid content and the second desired phenotype may comprise one or more of a morphological, stress tolerance, and production phenotype (or vice versa). As one example, the first desired phenotype may comprise a high cannabinoid content and the second desired phenotype may comprise plant vigor. In other embodiments, the first desired phenotype and the second desired phenotype may comprise variations of the same trait. For example, the first desired phenotype may comprise high cannabinoid content and the second desired phenotype may comprise low cannabinoid content (or vice versa).

In other embodiments, the first desired phenotype may be the same or similar to the second desired phenotype. As one example, in some embodiments, both the first and second cannabis plants may have photoperiod phenotypes or autoflowering phenotypes.

In other embodiments, the first and second cannabis plants may have at least one phenotype in common and at least one different phenotype. As one example, the first and second cannabis plants may each comprise a disease tolerance phenotype, while the first cannabis plant comprises high cannabinoid content and the second cannabis plant comprises low cannabinoid content (or vice versa).

In other embodiments, at least one of the first and second cannabis plants may be selected based on a first desired genotype and a second desired genotype, respectively. As used herein, “genotype” may refer to the genetic constitution of a plant. The genotype may comprise one or more of: the nucleic acid sequence of the entire genome, one or more chromosomes or portions thereof, one or more genes or portions thereof, and/or one or more genetic markers at specific loci within the genome. As used herein, “genetic marker” may refer to a polymorphic nucleic acid sequence or nucleic acid feature and any allele thereof. In some embodiments, the genotype may be determined by nucleic acid sequencing, polymerase chain reaction (PCR)-based techniques, hybridization assays such as DNA microarrays, restriction fragment length polymorphism (RFLP) analysis, genotyping-by-sequencing methods, or combinations thereof. Example methods for genotyping cannabis plants are described in U.S. patent application Ser. No. 15/735,825 (published as US20180171394), incorporated herein by reference. In other embodiments, the genotype may be determined using any other suitable techniques.

In some embodiments, the desired genotype may comprise one or more genetic markers associated with a desired phenotype. The term “associated with” in this context may refer to a genetic marker that tends to be inherited with the desired phenotype more often than expected by chance alone.

In other embodiments, the desired genotype may comprise a genotype of a specific variety or cultivar of cannabis known to produce the desired phenotype. In some embodiments, the desired genotype may comprise one or more genetic markers associated with a specific variety or cultivar of cannabis. The term “associated with” in this context may refer to a genetic marker that is found more often in one variety or cultivar than in any other variety or cultivar than would be expected by chance alone.

In some embodiments, at least one of the first and second desired genotype may comprise a genotype of a drug-type cannabis variety or cultivar. As used herein, “drug-type” may refer to a variety or cultivar of cannabis that is grown for use in a drug (or medicinal) and/or recreational application. The drug-type variety or cultivar may have at least a legal limit THC content. In some embodiments, the drug-type variety or cultivar has a THC content of at least about 0.3% THC by dry weight of the leaves and flowers. In some embodiments, the drug-type variety or cultivar has a THC content of at least about 0.2% THC by dry weight of the leaves and flowers. The drug-type variety or cultivar may have high or low CBD content.

In some embodiments, the drug-type variety or cultivar may be Cannabis sativa subsp. sativa (i.e. Cannabis sativa ), Cannabis sativa subsp. indica (i.e. Cannabis indica), or Cannabis sativa subsp. ruderalis (i.e. Cannabis ruderalis).

In some embodiments, the drug-type variety or cultivar may be a hybrid of two different subspecies/species. In some embodiments, the hybrid is a hybrid of Cannabis sativa subsp. sativa and Cannabis sativa subsp. indica. In some embodiments, the hybrid may be “balanced” such that approximately 50% of its genetic material is from Cannabis sativa subsp. sativa and approximately 50% of its genetic material is from Cannabis sativa subsp. indica. In other embodiments, the hybrid may be “sativa-dominant” such that over 50% of its genetic material is from Cannabis sativa subsp. sativa or “indica-dominant” such that over 50% of its genetic material is from Cannabis sativa subsp. indica.

Non-limiting examples of suitable drug-type varieties/cultivars include: “Blue Dream” (sativa-dominant hybrid); “Purple Kush” (Cannabis sativa subsp. indica); “Hindu Kush” (Cannabis sativa subsp. indica); “Agent Orange” (sativa-dominant hybrid); “Cheese Plant” (indica-dominant hybrid); and “Girl Scout Cookies” (indica-dominant hybrid).

In some embodiments, at least one of the first and second desired genotype may comprise a genotype of a hemp variety or cultivar. As used herein, “hemp”, “industrial hemp” or “fiber-type” may refer to a variety or cultivar of cannabis having less than a legal limit THC content (i.e. in some embodiments, less than about 0.3% THC by dry weight of the leaves and flowers and in other embodiments, less than about 0.2% THC by dry weight of the leaves and flowers). In some jurisdictions, industrial hemp must legally contain less than 0.2% or about 0.3% THC by dry weight of its leaves and flowers. The hemp variety or cultivar may have a high or low CBD content.

Non-limiting examples of suitable hemp varieties/cultivars include Piccolo (Picolo), Finola, X-59, CFX-2, CRS-1, and Canda. In some embodiments, the hemp variety or cultivar is dioecious. In other embodiments, the hemp variety or cultivar is monoecious.

In some embodiments, the first and second desired genotypes may be different. In some embodiments, the first desired genotype may be the genotype of a drug-type variety or cultivar and the second desired genotype may be the genotype of a hemp variety or cultivar (or vice versa). In other embodiments, the first desired genotype may be a first drug-type (or hemp) variety or cultivar and the second desired genotype may be a second drug-type (or hemp) variety or cultivar.

In other embodiments, the first and second genotypes may be the same or similar. For example, the first and second cannabis plants may be different individual plants of the same variety or cultivar. The individual plants may have similar genotypes but may have at least one different phenotypic trait due to small genetic variations and/or differences in the environmental conditions in which the plants were grown.

Other variations are also possible. In an alternative embodiment, a grafted plant may be provided comprising a scion from a cannabis plant and a rootstock from a non-cannabis plant. In some embodiments, the non-cannabis plant may be a plant of the Humulus genus, for example, Humulus lupulus (hops). In some embodiments, the non-cannabis plant may be a plant of the Urtica genus, for example, Urtica dioica(stinging nettle). The non-cannabis plant may be clonally propagated or seed-grown. In some embodiments, the non-cannabis plant may have any of the desired phenotypes described above with the exception of the biochemical phenotypes that are unique to cannabis plants.

As demonstrated in the Examples below, embodiments of the grafted cannabis plants described herein may exhibit at least one enhanced plant trait, harvest trait, or both, compared to clonally propagated cannabis plants. As used herein, a “plant trait” may be used synonymously with plant “phenotype” and may refer to a trait of a plant prior to harvesting. As used herein, a “harvest trait” may refer to a trait of a plant at the time of harvest or a characteristic of a harvested plant material. As used herein, “plant material” or “cannabis plant material” may refer to material which is obtained or derived from a plant or plant part. The plant material may be obtained from all or part of the scion, the rootstock, or the entire grafted cannabis plant. Non-limiting examples of enhanced plant traits that may be exhibited by the grafted plant include: enhanced plant vigor; enhanced stress tolerance; increased plant size and/or height; increased stem thickness; and increased floral yield. Non-limiting examples of enhanced harvest traits include: greater vegetative weight at harvest; greater weight of dried flowers at harvest; and one or more of higher cannabinoid content, THC content, CBD content, and terpene content of the harvested plant material.

In some embodiments, the grafted cannabis plant may exhibit one or more emergent properties. As used herein, an “emergent property” or an “emergent phenotype” may refer to a trait of a grafted plant that is not present in either of the first or second cannabis plants (from which the scion and rootstock are provided, respectively) or is substantially different than corresponding traits in either the first or second cannabis plants. In some embodiments, the emergent property may be substantially different than the first desired phenotype and the second desired phenotype. The emergent property may be due to interaction between the genotypes and/or phenotypes of the first and second cannabis plants, with or without the influence of an environmental component. As one example, the grafted plant may display a dwarf phenotype when neither the first or second cannabis plants are dwarfed.

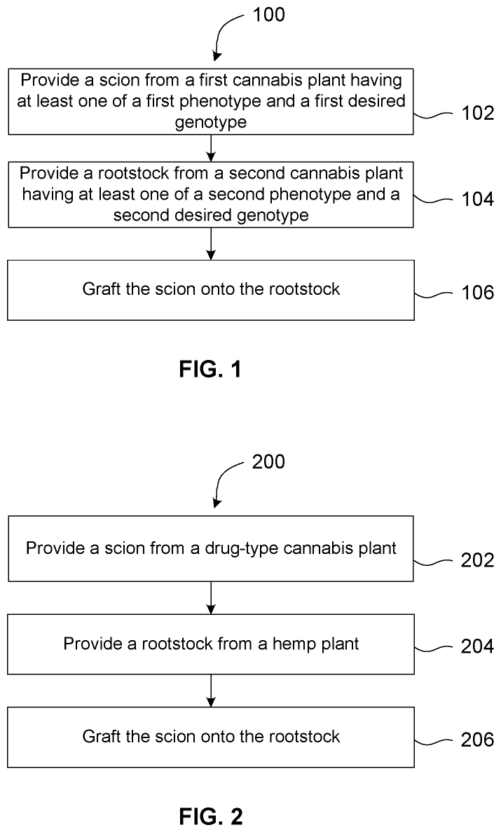

FIG. 1 is a flowchart of an example method 100 for producing a grafted cannabis plant, according to some embodiments. The method 100 may be used to produce embodiments of the grafted plants described herein.

At block 102, a scion may be provided from a first cannabis plant having at least one of a first desired phenotype and a first desired genotype. The term “providing”, when used in reference to providing a scion, may refer to taking a cutting of the first cannabis plant for use as the scion or to acquiring, buying, or otherwise obtaining the scion by any suitable means. The first desired phenotype and the first desired genotype may comprise any of the phenotypes or genotypes described above.

In some embodiments, the cutting selected for use as the scion may have a stem diameter approximately matching the stem diameter of the rootstock to be provided at block 104 as described below. In some embodiments, the stem of the scion may be cut at approximately a 45° angle or greater to match a cut of approximately the same angle on the rootstock as described below. In some embodiments, the cut may be performed 1-4 nodes below a shoot tip.

At block 104, a rootstock may be provided from a second cannabis plant having at least one of a second desired phenotype and a second desired genotype. The term “providing”, when used in reference to providing the rootstock, may refer to growing, acquiring, buying, or otherwise obtaining the rootstock by any suitable means. The second desired phenotype and the second desired genotype may comprise any of the phenotypes or genotypes described above.

To prepare the rootstock for grafting, the rootstock may be cut at a point along its stem and the top shoot discarded. In some embodiments, the rootstock may be cut at approximately a 45° angle or greater. In some embodiments, the cut may be between the cotyledon and the first true leaf or leaves. In other embodiments, the cut may be above the first true leaves. In other embodiments, the cut may be below the cotyledon.

At block 106, the scion may be grafted onto the rootstock. In some embodiments, grafting the scion onto the rootstock may comprise joining the scion and the rootstock together at respective cuts in their respective stems. In some embodiments, the scion and rootstock may be orientated for maximum surface area contact between the two cut surfaces. In some embodiments, the scion and rootstock may be held together by any suitable attachment means, for example, a grafting clip.

In alternative embodiments, the scion may be grafted onto the rootstock using any other suitable grafting technique, for example, splice grafting, saddle grafting, approach grafting, bud grafting, whip grafting, whip and tongue grafting, stub grafting, four-flap grafting, awl grafting, or veneer grafting.

The grafted plant may then be incubated under conditions to allow the scion and rootstock to form a vascular connection therebetween. In some embodiments, the grafted cannabis plant may be incubated in a dark, high-humidity environment immediately after grafting. In some embodiments, the grafted plant may be gradually introduced to increased light intensity and reduced humidity as the scion and rootstock interdigitate and the vascular connection is formed.

Once the vascular connection between the scion and the rootstock has substantially formed, the grafted cannabis plant may be transplanted to a suitable growth environment to allow for vegetative growth. For example, the grafted plant may be transplanted approximately 14 days after grafting. In some embodiments, the grafted cannabis plant may be transplanted to a flood tray under growth lights. In other embodiments, the grafted cannabis plant may be transplanted to a hydroponic culture room with growth lights. In some embodiments, the grafted plant may be grown under a suitable photoperiod to allow for vegetative growth, for example, an 18/6 photoperiod (about 18 hours of light, about 6 hours of dark).

In some embodiments, the grafted cannabis plant may be induced to flower. In some embodiments, where the scion and/or rootstock comprise a photoperiod phenotype, flowering may be induced by reducing the photoperiod under which the plants are grown. For example, the photoperiod may be reduced from an 18/6 photoperiod to a 12/12 photoperiod (about 12 hours of light, about 12 hours of dark). In other embodiments, where the scion and/or rootstock comprise an autoflowering phenotype, flowering may be induced by growing the plant for a suitable period of time until the onset of flowering.

Plant material may be harvested from the grafted cannabis plant at any suitable stage of growth. In some embodiments, plant material may be harvested when at least a portion of the leaf color fades to yellow and at least a portion of the pistils are browned. As one example, the floral material of the grafted cannabis plant may be harvested when greater than approximately 50% of the leaf color fades to yellow and approximately 80% pistil browning is achieved.

In some embodiments, the plant material harvested from the grafted plant may comprise at least one of: floral material, leaves, seeds, stems, stalks and combinations thereof. In some embodiments, the plant material to be harvested may comprise the floral material. The floral material may comprise at least one of flowers, flower buds, trichomes, and combinations thereof. The floral material may be harvested by trimming the flowers from the grafted cannabis plant using any suitable method. It will be understood that the floral material may also contain a portion of leaves, stems, and stalks harvested therewith.

In some embodiments, the plant material may be dried. The plant material may be dried using any suitable drying method, including but not limited to, air-drying or drying in a drying tumbler. Herein, dried floral material may also be referred to as “dried flower”.

FIG. 2 is a flowchart of another example method 200 for producing a grafted cannabis plant, according to some embodiments.

At block 202, a scion may be provided from a drug-type cannabis plant. The drug-type cannabis plant may be a plant of any of the drug-type varieties/cultivars described above. In some embodiments, the drug-type cannabis plant may be clonally propagated. In other embodiments, the drug-type cannabis plant may be seed-grown. In some embodiments, the drug-type cannabis plant may have any of the desired phenotypes described above. The scion may be provided from the drug-type cannabis plant in a similar manner as that described above at block 102 of method 100.

At block 204, a rootstock may be provided from a hemp plant, the rootstock compatible with the scion. The hemp plant may be a plant of any of the hemp varieties/cultivars described above. In some embodiments, the hemp plant may be clonally propagated. In other embodiments, the hemp plant may be seed-grown. In some embodiments, the hemp plant may have any of the desired phenotypes described above. The rootstock may be provided in a similar manner as described above at block 104 of method 100.

At block 206, the scion may be grafted onto the rootstock. The steps at block 206 may be similar to those at block 106 of method 100 as described above. The grafted plant may be grown to maturity and cannabis plant material may be harvested therefrom as described above.

Also provided herein is cannabis plant material obtained from any embodiment of the grafted plants described herein. In some embodiments, the cannabis plant material comprises dried flower.

Also provided herein is a cannabis-derived product generated from the cannabis plant material of any embodiment of the grafted plants described herein. The term “cannabis-derived product” in this context may refer to any substance comprising or produced from cannabis plant material. In some embodiments, the cannabis-derived product may comprise one or more of an extract, a resin, a concentrate, an isolate, and an oil. In some embodiments, the cannabis-derived product may further comprise a carrier oil. In some embodiments, the carrier oil comprises a medium-chain triglyceride (MCT) oil (e.g. coconut oil, vegetable oil, olive oil, etc.).

Also provided herein is a composition comprising the cannabis-derived product. In some embodiments, the composition may be in the form of a pharmaceutical product, a veterinary product, a natural health product, a dietary supplement, a food product, a beverage product, a cosmetic product, a topical product, a tincture, and/or a vaporizable product for use with a vaporizer.

In some embodiments, the composition may further comprise at least one pharmaceutically and/or nutritionally acceptable excipient. Non-limiting examples of suitable excipients include fillers, binders, carriers, diluents, stabilizers, lubricants, glidants, coloring agents, flavoring agents, coatings, disintegrants, preservatives, sorbents, sweeteners and any other pharmaceutically or nutritionally acceptable excipient. In other embodiments, the composition may further comprise any other ingredient or combination of ingredients.

Without any limitation to the foregoing, the grafted cannabis plants and methods for producing grafted cannabis plants are further described by way of the following examples. However, it is to be understood that these examples are for illustrative purposes only, and should not be used to limit the scope of the present disclosure in any manner.

### EXAMPLE 1

### Scion and Rootstock for Grafted Cannabis Plant

Scions were taken from the “Blue Dream” cultivar. The Blue Dream cultivar has a photoperiod phenotype and high-THC content. Cuttings for scions were taken from a mother plant grown under T5 fluorescent lighting set to an 18/6 vegetative photoperiod (18 hours on/6 hours off). For comparison, cuttings of the same mother plant were taken for cannabis clones at three time points (3, 7, and 14 days before date of grafting) and were clonally propagated in rockwool cubes (B'cuzz Roxx Multiplugs™; Atami B.V™.; Rosmalen, Netherlands).

Rootstocks were developed from the “Piccolo” (“Picolo”) industrial hemp cultivar. The rootstock hemp seed was sown into 72-cell flats containing potting mix (Sunshine Mix™ #4; Sun Gro Horticulture™; Agawam, Mass.) and controlled release fertilizer (Florikote™; Florikan™; Sarasota, Fla.). Rootstocks were grown for approximately 13 days under T5 fluorescent lights set to an 18 hours light/6 hours dark photoperiod.

### EXAMPLE 2

### Grafting Procedure

One day prior to grafting, irrigation to the rootstocks was restricted. Low rootstock soil moisture reduces transpiration and helps to maintain a connection between rootstock and scion.

For each rootstock, an approximately 45° or greater cut was performed between the cotyledon and first true leaf, and the top shoot was discarded. Scions were prepared for grafting by removing larger leaves and submersing in a 5% bleach solution for 60 seconds followed by rinsing in four water baths for 30 seconds each. Scions with matching stem diameter were selected for grafting and an approximately 45° or greater cut was performed 1-4 nodes below the shoot tip. The rootstock and scion were joined together with a 1.5 mm grafting clip (Royal Brinkman™; 's-Gravenzande, Netherlands) and were orientated for maximum surface area contact between the rootstock and scion.

The grafted plants were kept in a dark, high humidity healing chamber directly after grafting to reduce transpiration and allowed the rootstock and scion to successfully interdigitate. Grafted plants were placed in a 72-cell flat and were held approximately 2.5 cm above a tray containing water. Plants were misted thoroughly with water and a clear plastic dome (with vents closed) was affixed to the tray. The domed tray was carefully sealed within a black plastic bag, ensuring that water in the bottom tray did not contact the rootstock soil. The healing chamber (black plastic bag and domed tray) was held in the dark at room temperature for a minimum of 48 hours.

Twenty-four hours after grafting the plants were sprayed with water and returned to the healing chamber. Forty-eight hours after grafting, the plants were sprayed with water and returned to the healing chamber and the black plastic bag substituted with a clear plastic bag with a black cloth thereon to gradually introduce the plants to increased light intensity. The interior of the plastic bags was sprayed with water each time the domed tray was removed. Seventy-two hours after grafting the clear plastic bag was removed and the domed tray was placed under soft fluorescent light (5-15 μmol m² s⁻¹).

During the first three to five days after grafting, plants were misted with water several times each day and were gradually exposed to increased light intensity (15-40 μmol m² s⁻¹) and reduced humidity (70-85% relative humidity). After approximately ten days of gradually increasing light intensity and reducing humidity, misting with water was restricted and the vented dome was removed. Plants were closely monitored, and at the first sign of wilting, the dome was replaced until wilting subsided.

Approximately 14 days after grafting, both clones and grafts were transplanted to rockwool cubes (Delta™ 6.5; Grodan Inc.™ Milton, ON) and positioned on flood trays under grow lights.

### EXAMPLE 3

### Growth and Harvest of Grafted Cannabis Plants

Twelve grafted plants and twelve clones (from the same mother plant as the scions for the grafted plants) of similar height and developmental stages were selected for experimentation. Initially, plants were randomly assigned among two flood trays (60.5 cm×112 cm). Due to large differences in vigor in the early stages of the trial, the clones and grafts were derandomized and separated among two flood trays. Plants were evenly spaced on flood trays with an approximate plant density of 16 plants m⁻¹. Plants were grown under a 288 W full spectrum LED light (FusionBright™; Grow Lights Canada™; Beamsville, ON) set to an 18/6 photoperiod to promote vegetative growth. Lights were positioned approximately 15 cm above plants and adjusted according to increases in plant height during the trial. To induce flowering, a 12/12 photoperiod (12 hours on/12 hours off) was induced nine days after transplanting plants to rockwool cubes.

A flood-and-drain irrigation system was employed to irrigate plants to a depth of 3.5 cm for intervals of 2-3 minutes as required by clones and grafts. During the 18/6 photoperiod (18 hours on/6 hours off) for vegetative growth, trays were flooded with an all-purpose, water soluble nutrient solution (Peter's Excel Cal-Mag Special™; ICL Specialty Fertilizers™; Summerville, S.C.). During the 12/12 photoperiod (12 hours on/12 hours off) to induce flowering, trays were flooded with a nutrient solution formulated for flowering plants (MaxiBloom™; General Hydroponics™; Santa Rosa, Calif.). Applications of Intercept198 (imidacloprid; Bayer™; Leverkusen, Germany), Nova™ (myclobutanil; Dow AgroSciences™; Indianapolis, Ind.) and biological controls (Limonica™, Spidex™, Thripex-V198 and Spical™; Koppert Canada Ltd™; Surrey, BC) were applied for protection against thrips, powdery mildew and spider mites.

Plants were harvested when greater than 50% of canopy leaf colour faded to yellow and 80% pistil browning was achieved. Floral material was hand trimmed and allowed to dry for five to seven days after harvest. Samples of dried floral material from the six highest yielding grafts and clones were sent to a third-party testing lab (Anandia™; Vancouver, BC) for analysis of cannabinoid and terpene levels. All grafting, cloning, plant growth and postharvest drying were performed at 22-24° C.

### EXAMPLE

### Characterization of the Grafted Cannabis Plants

### Plant Traits

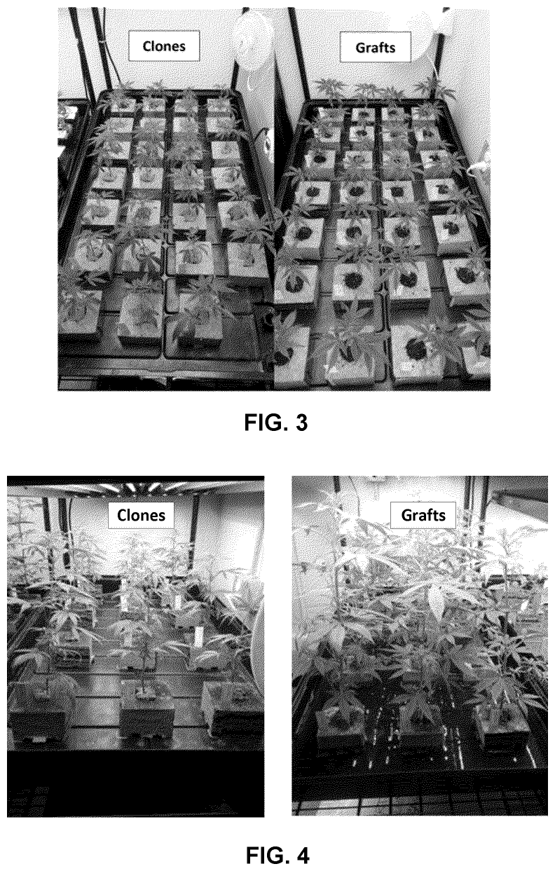

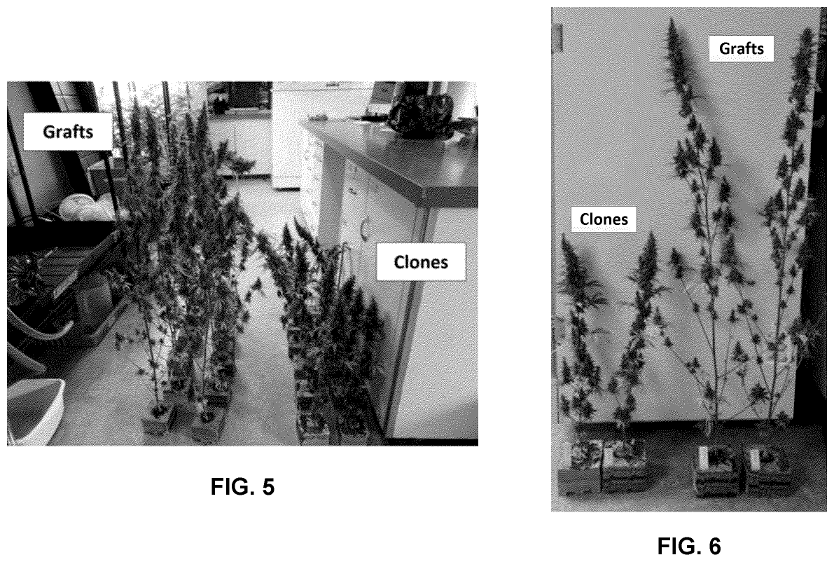

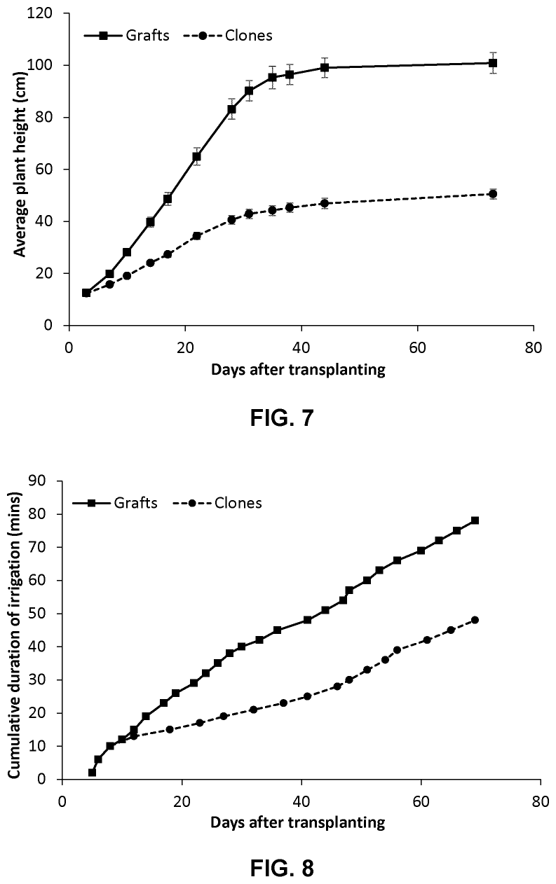

The grafted cannabis plants were compared to the clonally propagated cannabis plants from the same mother plant as the scions of the grafted plants. As shown in FIGS. 3 to 6, the grafted cannabis plants displayed increased height compared to clonally propagated cannabis plants at 15 days after grafting (FIG. 3), 12 days after induction of the 12/12 flowering photoperiod (12 hours on/12 hours off) (FIGS. 4), and 64 days after induction of the 12/12 flowering photoperiod (12 hours on/12 hours off) (FIGS. 5 and 6). As shown in FIG. 7, plant height was consistently higher for grafts than for clones over the course of the experiment, starting at approximately 7 days post-transplantation to harvest at 73 days post-transplantation. At harvest, final plant height for the grafted plants was 101 cm and for clones was 51 cm.

Grafted plants required more irrigation than cloned plants when irrigation was applied according to the moisture requirements of the media. As shown in FIG. 8, over the course of the experiment, clones required a total of 48 minutes of flood irrigation and grafts required a total of 78 minutes of flood irrigation.

Table 1 compares average plant traits of grafted cannabis plants compared to clones. Pistils emerged from both grafts and clones nine days after induction of a 12/12 photoperiod (12 hours on/12 hours off). During the main growth phases, clones had an average 10 leaves removed due to senescence and grafts had an average 43 leaves removed due to senescence. Similar differences between clones and grafts were observed for stem diameter, shoot length, primary shoot number and internode length. Stem diameter of grafts was an average of 17.4 mm thicker than clones. Shoot length was 19.1 cm longer for grafts than for clones. Leaf aspect ratio and primary shoot number were similar between clones and grafts, but internode length was larger for grafts than for clones.

TABLE 1

| Trait | Clones | Grafts |
|---|---|---|
| Time to first pistil emergence (days)^{a} | 9 | 9 |
| Stem diameter (mm) | 8.4 ± 0.9^{b} | 33.3 ± 2.8 |
| Internode length (mm) | 25.8 ± 2.2 | 54.4 ± 4.8 |
| Shoot length (cm) | 8.5 ± 0.6 | 27.7 ± 1.9 |
| Primary shoot number | 19.8 ± 0.6 | 22.3 ± 0.6 |
| Leaflet aspect ratio^{c} | 0.2 ± 0.015 | 0.2 ± 0.001 |
| Number of leaves pruned | 10.1 ± 1.1 | 42.7 ± 3.5 |

^{a}Time to pistil emergence is the duration of time after induction of a 12/12 photoperiod (12 hours on/12 hours off) until first appearance of pistils.
^{b}Values following ‘±’ indicate the standard error for each plant trait mean.
^{c}Leaflet aspect ratio is the leaflet width divided by leaflet length and averaged for three leaves.

### Harvest Traits

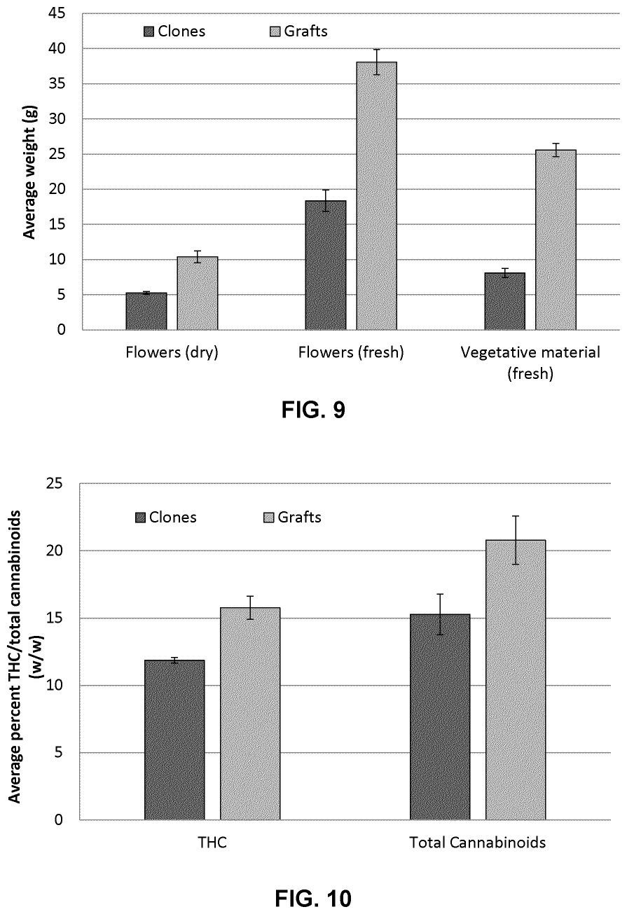

Mature flowers of clones and grafts were morphologically similar. As shown in FIG. 9, average vegetative weight (fresh), floral weight (fresh) and floral weight (dry) were greater for grafts than for clones. The average weight of vegetative material for each clone was 8.1 g and for each graft was 25.6 g. The average fresh weight of flowers for clones was 18.4 g plant⁻¹ and for grafts was 38.1 g plant⁻¹. Average weight of dried flowers was higher for grafts than for clones (grafts=10.4 g plant⁻¹, clones=5.3 g plant⁻¹).

As shown in FIG. 10, total THC equivalents (concentration of Δ⁹-tetrahydrocannabinol and Δ⁹-tetrahydrocannabinolic acid) significantly differed between grafts and clones. The dry flowers of clones averaged 11.9% THC and the grafts averaged 15.8% THC. Concentration of total cannabinoids and terpenes were both higher for grafts than for clones. Percent total cannabinoids was higher for the dried flowers of grafts than for clones. In comparison to clones, the grafts averaged 4.85% higher total cannabinoid levels, representing a relative 32% increase in total cannabinoid content over the clones.

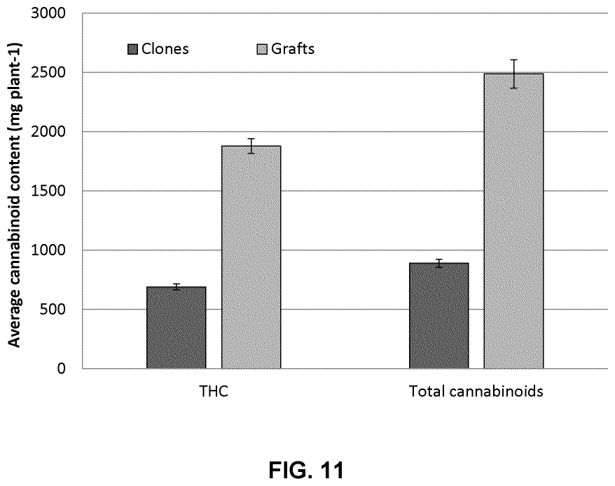

As shown in FIG. 11, when accounting for flower yield, per-plant THC content was much higher for grafts than for clones. Each graft attained an average of 1879 mg THC per plant while clones averaged approximately 692 mg THC per plant. Similarly, total cannabinoid content per plant was much greater for grafts than clones, with grafts yielding an average of 2489 mg of cannabinoids per plant.

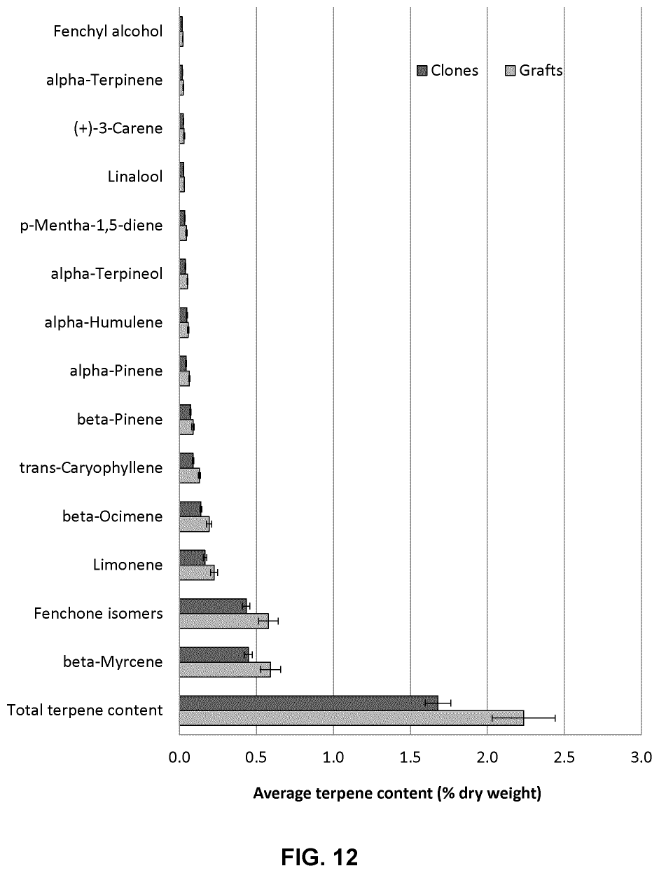

As shown in FIG. 12, in addition to higher cannabinoid content, the concentration of terpenes for grafts was higher than clones. For all the terpenes identified by gas chromatography and mass-spectrometry, the grafts possessed a higher concentration than clones. The average total terpene content of grafts was 2.24% and the total terpene content of clones was 1.68%, indicating a relative 33% increase in terpene concentration for grafted cannabis plants.

### EXAMPLE 4

### Grafting Experiments with Purple Kush Cannabis Cultivar

Scions were taken from “Purple Kush” (PK), a high-THC, drug-type cannabis cultivar. Clonally propagated PK was compared to PK grafted on to “Piccolo” (“Picolo”) hemp rootstocks. Rootstocks were obtained from seed-derived “Piccolo” (“Picolo”) hemp plants. Cuttings from a PK cannabis plant were used to develop clonally propagated plants and for grafting scions onto hemp rootstocks. Cuttings for clones were rooted in rockwool cubes over a two-week period. The grafting procedure was similar to that described above with respect to the Blue Dream cultivar.

Eight clones and grafts were grown to physiological maturity and harvested. Total plant weight and floral weight was assessed at harvest. Floral material was dried at room temperature and dried samples were sent to Anandia™ for laboratory testing and analysis of cannabinoid composition.

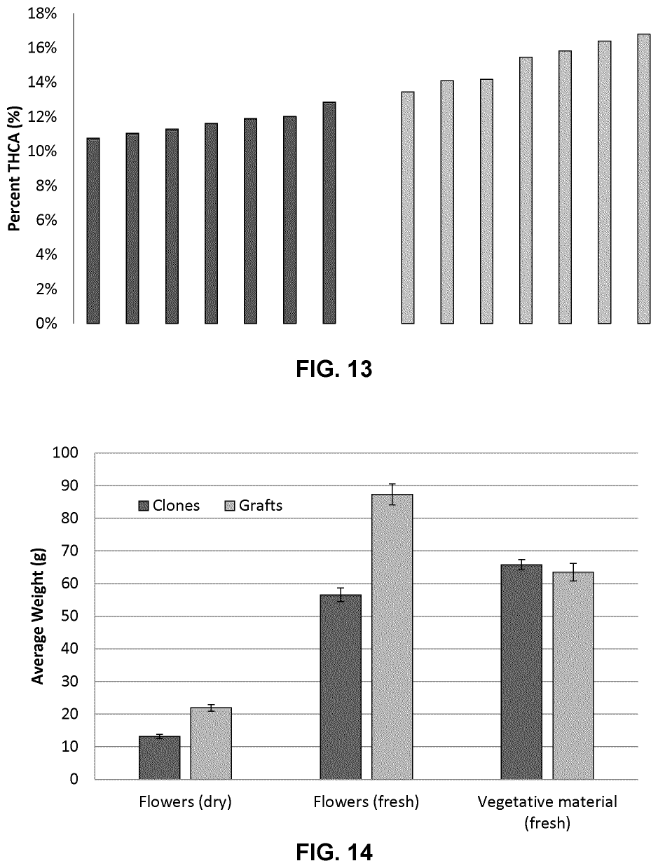

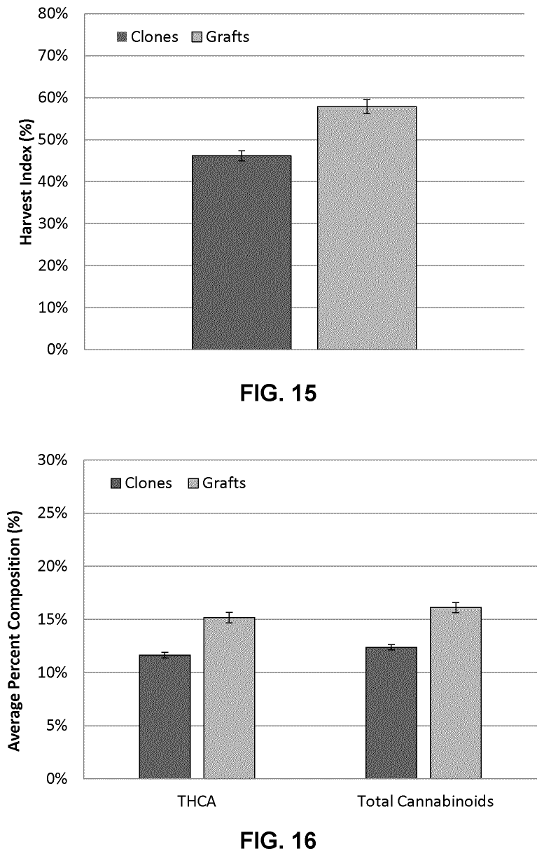

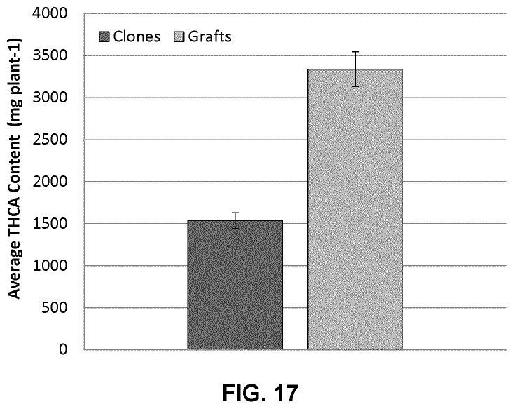

All grafted plants obtained higher THCA content than clonally propagated plants (FIG. 13). Average floral fresh weight was 55% greater for grafted PK plants than for clonally propagated plants (FIG. 14). Harvest index, the proportion of total plant weight that is floral material, was higher for grafted plants than for clones (FIG. 15). Average total cannabinoid content was approximately 3.6% greater for grafted plants than for the floral material of clonally propagated plants (FIG. 16). Total THCA content per plant (mg plant⁻¹) was greater for grafted cannabis than clonally propagated cannabis (FIG. 17).

### EXAMPLE 5

### Grafting Experiments with Other Cannabis Cultivars

Grafted experiments were also conducted using scions from other high-THC, drug-type cannabis cultivars. Scions were taken from “Hindu Kush” (Cannabis sativa subsp. indica), “Agent Orange” (sativa-dominant hybrid), “Cheese Plant” (indica-dominant hybrid), and “Girl Scout Cookies” (indica-dominant hybrid) and grafted onto respective “Piccolo” (“Picolo”) hemp rootstocks. All of the drug-type cultivars have photoperiod phenotypes and were clonally propagated. The grafting procedures were similar to that described above with respect to the Blue Dream grafted plants. All scions were successfully grafted with “Piccolo” (“Picolo”) rootstocks.

It should be apparent to those skilled in the art that more modifications besides those already described are possible without departing from the inventive concepts herein. The inventive subject matter, therefore, is not to be restricted except in the scope of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although particular embodiments have been shown and described, it will be appreciated by those skilled in the art that various changes and modifications might be made without departing from the scope of the invention. The terms and expressions used in the preceding specification have been used herein as terms of description and not of limitation, and there is no intention in the use of such terms and expressions of excluding equivalents of the features shown and described or portions thereof, it being recognized that the invention is defined and limited only by the claims that follow.

It is to be understood that a combination of more than one of the approaches described above may be implemented. Embodiments are not limited to any particular one or more of the approaches, methods or apparatuses disclosed herein. One skilled in the art will appreciate that variations, alterations of the embodiments described herein may be made in various implementations without departing from the scope of the claims.

1. A method for producing a grafted cannabis plant, the method comprising:
providing a scion from a drug-type cannabis plant having at least one of a first desired phenotype and a first desired genotype;
providing a rootstock from a hemp plant having a legal limit THC content of less than about 0.3% THC by dry weight, the hemp plant having at least one of a second desired phenotype and a second desired genotype, the rootstock compatible with the scion; and
grafting the scion onto the rootstock.

2. The method of claim 1, wherein the first desired phenotype comprises at least one of a first biochemical phenotype, a first morphological phenotype, a first photoperiod or autoflowering phenotype, and a first production phenotype.

3. The method of claim 2, wherein the first biochemical phenotype comprises a desired cannabinoid content.

4. The method of claim 3, wherein the desired cannabinoid content comprises at least one of high THC content, high CBD content, and high cannabinoid content.

5. The method of claim 2, wherein the first morphological phenotype comprises at least one of plant size, plant height, stem thickness, flower number, flower size, flower color, flower density, trichome density, branching number, branching degree, branching architecture, and internode length.

6. The method of claim 2, wherein the first production phenotype comprises at least one of growth rate, yield, stress tolerance, and plant vigor.

7. The method of claim 2, wherein the second desired phenotype comprises as least one of a second biochemical phenotype, a second morphological phenotype, a second photoperiod or autoflowering phenotype, and a second production phenotype.

8. The method of claim 7, wherein the second morphological phenotype comprises at least one of plant size, plant height, stem thickness, root structure morphology, root thickness, and rooting quality.

9. The method of claim 7, wherein the second production phenotype comprises at least one of growth rate, yield, stress tolerance, plant vigor, and nutrient uptake.

10. The method of claim 7, wherein the first desired phenotype comprises the first biochemical phenotype and the second desired phenotype comprises at least one of the second morphological phenotype and the second production phenotype.

11. (canceled)

12. The method of claim 1, wherein the drug-type cannabis plant comprises a Cannabis sativa L. subspecies sativa or Cannabis sativa L. subspecies indica variety or cultivar.

13. The method of claim 1, wherein the drug-type variety or cultivar is cannabis plant comprises a hybrid of variety or cultivar Cannabis sativa L. subspecies sativa and Cannabis sativa L. subspecies indica.

14. (canceled)

15. The method of claim 1, wherein at least one of the drug-type cannabis plant and the hemp plant is clonally propagated.

16. The method of claim 1, wherein at least one of the drug-type cannabis plant and the hemp plant is seed-grown.

17. The method of claim 1, wherein the drug-type cannabis plant is clonally propagated and the hemp plant is seed-grown.

18. The method of claim 1, wherein the drug-type cannabis plant is seed-grown and the hemp plant is clonally propagated.

19. A grafted cannabis plant produced by the method of claim 1.

20. The grafted cannabis plant of claim 19, wherein the grafted plant exhibits at least one of: an enhanced plant trait, an enhanced harvest trait, and an emergent property.

21. The grafted cannabis plant of claim 20, wherein the emergent property is not present in the drug-type cannabis plant or the hemp plant.

22. The grafted cannabis plant of claim 20, wherein the emergent property is substantially different than the first desired phenotype and the second desired phenotype.

23-49. (canceled)
